(19) United States
(12) Patent Application Publication
(10) Pub. No.: US 2014/0081169 A1
(43) Pub. Date: Mar. 20, 2014

(54) SYSTEM AND METHOD OF TETHERLESS INSUFFLATION IN COLON CAPSULE ENDOSCOPY

(71) Applicant: Vanderbilt University, Nashville, TN (US)

(72) Inventors: Jason Gerding, Nashville, TN (US); Byron Smith, Memphis, TN (US); Keith L. Obstein, Nashville, TN (US); Pietro Valdastri, Nashville, TN (US)

(73) Assignee: Vanderbilt University, Nashville, TN (US)

(21) Appl. No.: 14/029,687

(22) Filed: Sep. 17, 2013

Related U.S. Application Data
(60) Provisional application No. 61/702,178, filed on Sep. 17, 2012.

(51) Int. Cl. A61M 13/00; A61B 1/04; A61K 31/194

(52) U.S. Cl. CPC A61M 13/003; A61K 31/194; A61B 1/041
USPC 600/560

(57) ABSTRACT

A system and method of wireless controlled CO₂ insufflation for use in colon capsule endoscopy. The system includes a device to inflate the colon through the use of a swallowable capsule including a first compound and a second compound for generating a biocompatible chemical reaction that provides a level of insufflation to enhance visualization and to allow for magnetic locomotion within the colon. The chemical reaction achieves relevant colon insufflation (enough to enable diagnostic relevance) by producing CO₂ (carbon dioxide).

### RELATED APPLICATIONS

This application claims the benefit of U.S. Provisional Patent Application No. 61/702,178, filed on Sep. 17, 2012, the entire contents of which are incorporated herein by reference.

### BACKGROUND OF THE INVENTION

While little has changed with semi-flexible endoscopes since they were first made possible by the introduction of glass fibers in 1957, a great deal of effort has been put forth on the development of accessories for endoscopes. Through the use of the endoscope's accessory port, physicians can deploy spectroscopy-based diagnostic measures, deliver hemostasis-promoting therapies, take biopsies, or remove large volumes of tissue including precancerous polyops or advanced carcinomas. In an effort to advance the field of wireless capsule endoscopy (WCE), researchers throughout the world are working to develop many of these same capabilities on-board capsule-based platforms (for example, capsule-based spectroscopy, capsule-based delivery of clips for hemostasis, and capsule-based biopsy). And while many of these modalities have been proven feasible in a single-capsule or multi-capsule platform, their implementation often requires (or at least would benefit from) the ability to insufflate the intestine.

The ability to inflate the intestine makes an endoscopist's job much easier. Rather than navigate through, and operate within, the compliant folds of the large intestine, the ability to distend tissue through the use of a pressurized gas or liquid provides the endoscopist with an enhanced view of the endoscope's surroundings and a greater ability to move within said surroundings. In an effort to provide this same ease of motion and enhanced visualization to WCE, the present invention is directed to a capsule-based platform that, when remotely activated, can deliver a volume of gas sufficient for enhancing local visualization and freedom of movement.

### SUMMARY OF THE INVENTION

Robotic mechanisms promise to enhance the diagnostic abilities of capsule endoscopes, endow them with novel interventional capabilities and reduce the invasiveness of endoscopy. The success of traditional endoscopy in diagnosing disease of the gastrointestinal (GI) tract can be attributed to the clear view that such techniques provide of the intestinal lumen and the range of motion they are capable of displaying. When viewed in the context of capsule endoscopy, the ability to clearly view tissue and navigate within the GI track both depend on the ability to distend tissue.

With capsule endoscopy becoming a cornerstone for evaluation of the small intestine, implementing this technology successfully for evaluation of the human colon has been challenging due to the need for safe, controlled, reliable insufflation. Wireless insufflation looks to enhance wireless capsule endoscopy by enhancing visualization and, in the case of magnetic locomotion, enhancing mobility. Carbon dioxide (CO₂) for the purpose of colonic insufflation has been found to be advantageous over traditional air insufflation since CO₂ is readily absorbed via the colon, thereby reducing patient discomfort due to the effect of colonic distention.

Capsule endoscopy (CE) allows a physician to view the interior lining of a patient's colon. However, in the case of CE, the physician's view of the colon consists of thousands of still images taken from a camera embedded within a swallowable capsule. This imaging technique not only results in sharper image quality (than virtual colonoscopy), it also holds promise for providing physicians with a real-time method for exploring the colon. While commercially available capsule endoscopes currently only serve as passive observers, a growing body of research is showing how these devices might one day allow physicians to precisely control the position and orientation of capsule endoscopes and even provide therapeutic capabilities.

In one application, the present invention can be used for colorectal cancer screening. Colorectal cancer (CRC) is a proven killer that affects one in five Americans. In 2012 alone, CRC is expected to take the lives of 51,690 Americans.

The present invention relates to a novel system of wireless controlled CO₂ insufflation for use in colon capsule endoscopy. In particular, the present invention is a wireless system to inflate the colon through the use of a biocompatible chemical reaction that provides a level of insufflation to enhance visualization and to allow for magnetic locomotion within the colon. These chemical formulations achieve relevant colon insufflation (enough to enable diagnostic relevance) by producing CO₂ (carbon dioxide) starting from chemical reactants that can be integrated into a swallowable capsule.

The biocompatible chemical reactions can include acetic acid+sodium bicarbonate, Citric acid+sodium bicarbonate, Acetic acid+potassium bicarbonate, Citric acid+potassium bicarbonate, Aluminum Sulfate+sodium bicarbonate, Aluminum Sulfate+potassium bicarbonate, Acetic acid+sodium bicarbonate+Carbonic anhydrase, Citric acid+sodium bicarbonate+Carbonic anhydrase, Acetic acid+potassium bicarbonate+Carbonic anhydrase, Citric acid+potassium bicarbonate+Carbonic anhydrase, acetic acid+sodium carbonate, Citric acid+sodium carbonate, Acetic acid+potassium carbonate, Citric acid+potassium carbonate.

The proposed solution entails the use of sodium bicarbonate and citric acid. This reaction achieved a volume of gas that has been found to be sufficient to distend the colon lumen. This chemical reaction also generates an inflation that produces a tangible enhancement to visualizing the colon lining.

Carbon dioxide is the product responsible for inflation and is produced by the reaction of potassium bicarbonate and citric acid. CO₂ is easily absorbed through the internal mucosa into the blood, and its use avoids overdistention and post-procedure abdominal discomfort. The reaction between potassium bicarbonate and citric acid has been found to generate the largest output of CO₂. However, sodium bicarbonate and citric acid is preferred for human use as potassium bicarbonate may result in complications for patients with renal failure.

In some embodiments of the devices described below, a first compound such as citric acid is in a first chamber, and a second compound such as sodium bicarbonate is in a second chamber. The first compound may be in solid form or in solution. Similarly, the second compound may be in solid form or in solution.

In some embodiments, a molar ratio of the first compound to the second compound is about 1:1. In other embodiments, the molar ratio of the first compound to the second compound is about 2:1. In further embodiments, the molar ratio of the first compound to the second compound is about 3:1. In still further embodiments, the molar ratio of the first compound to the second compound is about 4:1. Preferably, the molar ratio of the first compound to the second compound is between about 4:1 to about 2:1. Even more preferably, the molar ratio of the first compound to the second compound is about 3:1.

More particularly, in some embodiments, a molar ratio of the citric acid to the sodium bicarbonate is about 1:1. In other embodiments, the molar ratio of the citric acid to the sodium bicarbonate is about 2:1. In further embodiments, the molar ratio of the citric acid to the sodium bicarbonate is about 3:1. In still further embodiments, the molar ratio of the citric acid to the sodium bicarbonate is about 4:1. Preferably, the molar ratio of the citric acid to the sodium bicarbonate is between about 4:1 to about 2:1. Even more preferably, the molar ratio of the citric acid to the sodium bicarbonate is about 3:1.

The reactions used have the potential to obscure the view from a capsule endoscope of the colon due to the production of foam. As a result, the foam may be used to disperse dyes in a manner akin to chromoendoscopy. In such a case, the reactants could be pre-mixed with indigo carmine to allow an IRC to release dye-infused foam throughout the colon prior to inspection with a WCE. Studies concerning the clinical relevance of chromoendoscopy have reported very site dependent results, indicating that the technique may depend considerably on the operator. It therefore stands to reason that incorporating chromoendoscopy in a robotic-based WCE platform could remove operator dependencies and provide the advantage associated with chromoendoscopy to a larger number of patients.

In one embodiment, the invention provides a device for insufflating a body cavity. The device comprises a first chamber, a second chamber, and a port; a wall between the first chamber and the second chamber, the wall including a recessed portion and an opening in the recessed portion for fluid communication between the first chamber and the second chamber; a magnetic sphere positioned in the first chamber and configured to be received in the recessed portion and to close the opening; and a ferromagnetic ring positioned in the second chamber, the ferromagnetic ring including an opening aligned with the opening in the recessed portion, the ferromagnetic ring magnetically coupled to the magnetic sphere.

In another embodiment the invention provides a device for insufflating a body cavity. The device comprises a first chamber including citric acid; a second chamber including sodium bicarbonate; a port in one of the first chamber and the second chamber; and a mixing chamber that combines the sodium bicarbonate and the citric acid to produce a sufficient amount of carbon dioxide (CO₂) through the port to insufflate the body cavity upon activation of the device.

In another embodiment the invention provides a method of insufflating a body cavity. The method comprises positioning a swallowable device in a body cavity; activating the device within the body cavity by applying a magnetic field near the device; admixing sodium bicarbonate and citric acid in the capsule; and producing a sufficient amount of carbon dioxide (CO₂) due to the chemical reaction between the sodium bicarbonate and citric acid to insufflate the body cavity.

Other aspects of the invention will become apparent by consideration of the detailed description and accompanying drawings.

### BRIEF DESCRIPTION OF THE DRAWINGS

FIG. 1 is a perspective view and a cross-sectional view of a wireless capsule device according to an embodiment of the present invention.

FIG. 2 is a disassembled view of the wireless capsule device illustrated in FIG. 1.

FIG. 3 illustrates an experimental setup used during ex vivo assessment of the wireless capsule devices disclosed herein.

FIG. 4 illustrates internal views of the colon (top) and external views of the colon (bottom) following activation of the wireless capsule device shown in FIGS. 1-2 during experimentation.

FIG. 5 illustrates foam escaping from the wireless capsule device shown in FIGS. 1-2 (left) and obstructing the view of the colon wall (right).

FIG. 6 is a cross-sectional view of a wireless capsule device according to an embodiment of the present invention.

FIG. 7 is a disassembled view of the wireless capsule device illustrated in FIG. 6.

FIG. 8 is a cross-sectional view of a wireless capsule device according to an embodiment of the present invention.

FIG. 9 is a disassembled view of the wireless capsule device illustrated in FIG. 8.

FIG. 10 is a disassembled view of a wireless capsule device according to an embodiment of the present invention.

FIG. 11 is a graphical representation of transient output produced by the wireless capsule device shown in FIG. 10 with average initial mass of potassium bicarbonate equal to 0.8 grams and an initial volume of 0.9 mL of citric acid solution having a mass concentration of 1.5 g/mL.

FIG. 12 is an endoscopic view (left) and an external view (right) demonstrating the level of insufflation provided by one of the wireless capsule devices shown in FIG. 10 approximately two minutes after activation.

FIG. 13 is an endoscopic view (left) and an external view (right) demonstrating the level of insufflation provided by three of the wireless capsule devices shown in FIG. 10 approximately one minute after activation.

FIG. 14 is an endoscopic view (left) and an external view (right) showing a secondary activation provided by three of the wireless capsule devices shown in FIG. 10 approximately two minutes after initial activation during the same ex vivo trial.

FIG. 15 is an endoscopic view (left) and an external view (right) demonstrating the level of visualization provided by three of the wireless capsule devices shown in FIG. 10 approximately one minute after a secondary activation.

FIG. 16 illustrates (a) an image from a colonoscope of the colon prior to insufflation, (b) an image after insufflation. Note that in this image much more of the intestinal surface can be seen. Also pictured here is a prototype of a capsule robot with legs, (c) an image showing a capsule controlled by external magnetic fields that has difficulty moving through collapsed intestinal folds, (as do other capsules with active locomotion).

FIG. 17 illustrates the experimental setup for a feasibility test conducted to determine how much insufflation is required to improve visualization within the large intestine. Ex vivo porcine intestine was arranged in a phantom model to simulate the shape of the human colon within the abdomen.

FIG. 18 illustrates the results of the first feasibility test at different inflation increments. (a) The intestine in its deflated state with no markers visible. (b) With just 50 mL of insufflation, four of the nine markers became visible. (c) At 200 mL, all nine markers first come into the field of view. (d) The threshold above which all nine markers were consistently visible was 450 mL. (e) The intestine in its fully inflated state at 1500 mL of insufflation.

FIG. 19 are pictures of a locomotion experiment at different inflation increments. (a) The intestine in its deflated state, where no capsule motion was possible. (b) With just 50 mL of insufflation, the capsule moved an average distance of 66.7 mm. (c) At 100 mL, the capsule moved an average distance of 150 mm. (d) At 150 mL, the capsule moved an average distance of 188.3 mm. (e) At 200 mL, the capsule moved an average distance of 243.3 mm. (f) At 250 mL, the capsule was able to move the entire length of the colon (300 mm), with an average distance of 295 mm.

FIG. 20 is a graphical representation of the distance the capsule traveled (mm) at each inflation increment.

FIG. 21 is a picture of the experimental setup for reacting known volumes of H₂O₂ to measure gas production. A silver screen catalyst was dropped into the mixing flask which held a small initial volume of H₂O₂. The gas generated displaced water in the holding flask which was measured in a graduated cylinder.

FIG. 22 is a graphical representation of the volume of gas produced (mL) for initial amounts of liquid H₂O₂ at 30%, 50%, and 70% concentrations by mass.

FIG. 23 is a graphical representation of the maximum temperature recorded directly underneath the catalyst screen during the decomposition reaction.

FIG. 24 is a graphical representation of theoretical output from selected acid/base combinations.

FIG. 25 is a graphical representation of theoretical predictions and experimental observations for the output produced by given acid/base combinations within the first fifteen minutes when an initial volume of reactants equal to 1.0 mL is reacted in the presence of 0.5 mL of H₂O.

FIG. 26 is a graphical representation of transient output generated by various acid/base combinations when initial volumes of the reactants are set equal to 1.5 mL.

FIG. 27 is a graphical representation of transient output generated by citric acid and potassium bicarbonate when initial volumes of the reactants are set equal to 1.5 mL.

FIG. 28 is a graphical representation of transient output generated by citric acid and potassium bicarbonate when initial volumes of the reactants and water are set equal and total initial volumes are 1, 1.5 and 2 mL.

FIG. 29 is a graphical representation of the average output generated within fifteen minutes by citric acid and potassium bicarbonate when the ratio of initial volumes between water and reactants is set equal 0.5/1 for the cases of total initial volumes equal 1, 1.5 and 2 mL.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like.

Preliminary efforts of wireless capsule designs investigated the catalytic decomposition of hydrogen peroxide in addition to a number of effervescent reactions for use as possible gas generators in a wireless capsule insufflation platform. While hydrogen peroxide was found to have an excellent expansion ratio, recent findings published in the Journal of Clinical Gastroenterology and Hepatology have shown that even concentrations on par with the weakest solutions can result in serious damage when ingested.

Specifically, the designs presented herein utilize citric acid (C₅H₈O₇) and potassium bicarbonate (KHCO₃) to generate carbon dioxide (CO₂), however as noted above, the device designs preferably use sodium bicarbonate and citric acid. For citric acid reacted with sodium bicarbonate the stoichiometric ratio is three moles citric acid to one mole sodium bicarbonate. For citric acid with potassium bicarbonate the stoichiometric ratio is three moles citric acid to one mole of potassium bicarbonate. Based on the studies performed with citric acid and sodium bicarbonate, citric acid being in solution and sodium bicarbonate being a powder, a solution of 1.5 g/mL gave the best compromise between rate of reaction (the reaction needs to occur quickly so the doctor isn't waiting around) and total output (there is only so much space in the capsule so we need to get the most CO₂ possible so the patient doesn't have to swallow many pills).

Based on a discussion below, one or more capsules should be capable of providing approximately 450 mL to locally enhance visualization, or, as little as 250 mL to enhance locomotion in a section of colon approximating the length of the longest straight portion of the human colon.

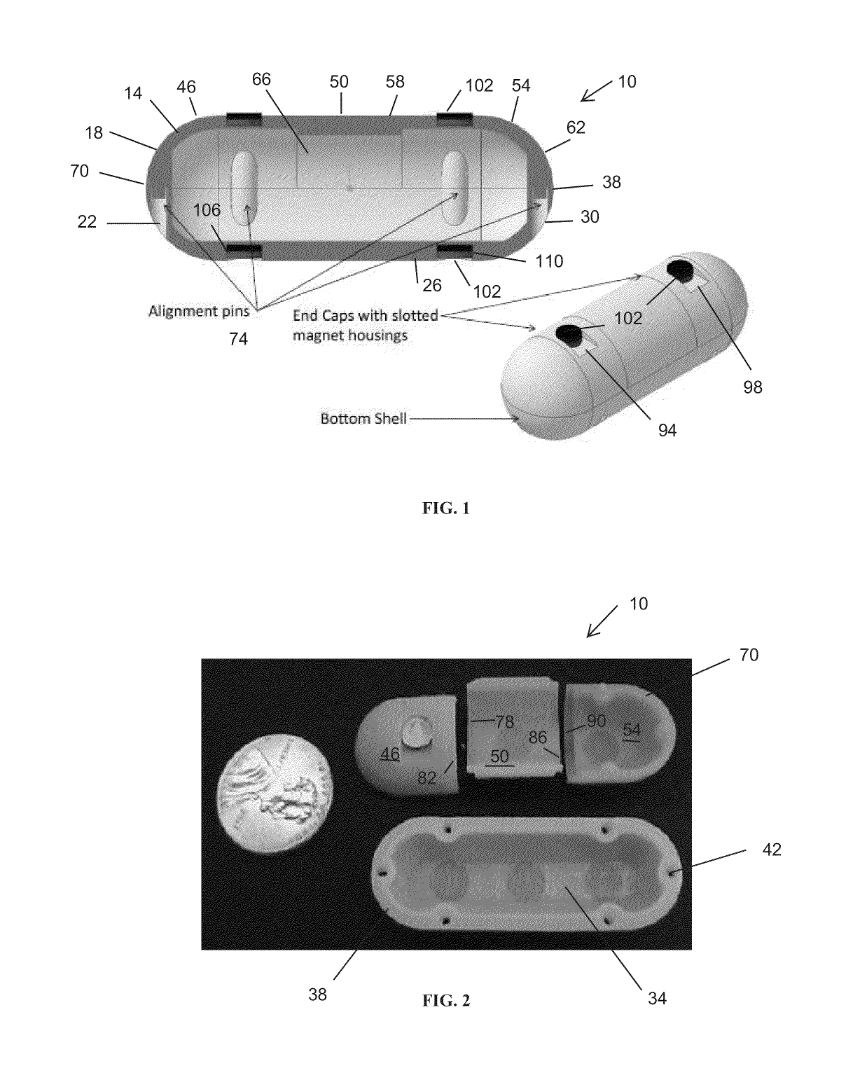

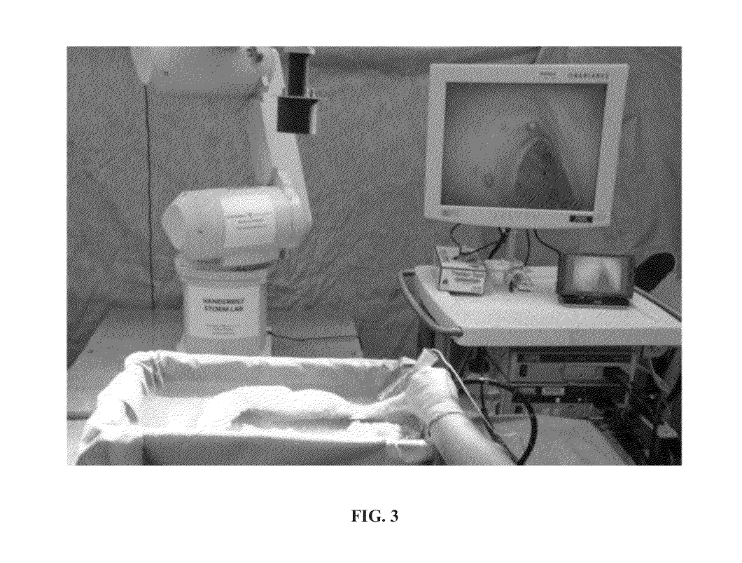

The wireless capsule device of the present invention is based on the specifications for relevant volumes of gas needed to enhance visualization and locomotion within the colon as discussed below. FIGS. 1-2 illustrate a wireless capsule device 10 according to an embodiment of the present invention. In the first concept, the device 10 carries a payload comprised of a first compound (e.g., a powdered reactant). In this embodiment, the first compound can be in solid form or in solution. In one particular embodiment, the first compound is sodium bicarbonate is solid form. When activated, the device 10 is configured to break apart, exposing its contents to the fluid found in the colon. This concept allows the device 10 to maximize achievable output by mitigating the need to carry water. This device 10 has an advantage of being able to transport a larger volume of reactants for a given capsule volume, since it does not require that H₂O be carried onboard, it does not provide the means to start and stop gas production.

The wireless capsule device 10 includes a housing 14 comprised of a first upper half section 18 and a second lower half section 22. The second lower half section 22 includes a bottom wall 26 and a sidewall 30 thereby defining a recess 34 having a periphery defining an outer edge 38 in an oval shape. The sidewall 30 has a thickness suitable for including a plurality of alignment features 42. The alignment features 42 illustrated in FIG. 2 are holes or recesses.

The first upper half section 18 is comprised of a first side portion 46, a middle portion 50, and a second side portion 54. The first upper half section 18 includes a top wall 58 and a sidewall 62 thereby defining a recess 66 having a periphery defining an outer edge 70 in an oval shape. The outer edge 70 is substantially equal (or equal) in dimensions as the outer edge 38 of the second lower half section 22 such that the first upper half section 18 and the second lower half section 22 can be coupled together and define a volume therebetween for holding fluid and/or chemical reactants (e.g., solid material).

The sidewall 62 of the first side portion 46 and the second side portion 54 have a thickness suitable for including a plurality of alignment features 74 configured to couple with the alignment features 42 in the second lower half section 22. The alignment features 74 illustrated in FIG. 2 are pins, pegs or posts configured to be received in the holes or recesses. The sidewall 62 of the middle portion 50 and the respective portion of the sidewall 30 on the second lower half section 2 do not include alignment features 42, 74.

The middle portion 50 of the first upper half section 18 includes a first U-shaped edge 78 configured to couple to a complementary U-shaped edge 82 on the first side portion 46. The middle section 50 also includes a second U-shaped edge 86 configured to couple to a complementary U-shaped edge 90 on the second side portion 54. These U-shaped edges include mating features that align and constrain the middle portion 50.

The top wall 58 of the first side portion 46 includes a recess 94, and the second side portion 54 includes a recess 98. The recesses 94, 98 are configured to receive and retain a permanent magnet 102. The bottom wall 26 of the second lower half section 22 includes a first recess 106 generally aligned with the recess 94 of the first side portion and a second recess 110 generally aligned with the recess 98 on the second side portion 54. The recesses 106, 110 are configured to receive and retain a permanent magnet 102.

These two sets of magnets 102 form a magnetic link between the first upper half section 18 and the second lower half section 22 to create a seal therebetween. The device 10 can be activated by introducing an external magnetic field strong enough to overcome the force generated by the magnetic coupling that exists between the two sets of magnets 102. When the magnetic coupling of the two sets of magnets 102 is overcome, the seal is released and fluid from the surroundings is free to enter the device 10. The fluid from the surroundings contacts a first compound (e.g., a base such as, for example, sodium bicarbonate in solution or in solid form) to generate a chemical reaction between the fluid (e.g., citric acid in solution or in solid form) and the first compound. The onset of the reaction generates pressure which serves to further open the device 10, thereby allowing the contents of the device 10 to become exposed to fluid found in the colon.

Ex-Vivo Trials Using Device 10

Ex vivo trials were performed to obtain qualitative results from the reaction between potassium bicarbonate and citric acid. As noted below, this reaction resulted in the best solution in terms of yield of gas within the considered time interval. The aim of these trials is the qualitative evaluation of colon lining visualization, as a measure of the accomplishment of the insufflation. The evaluation was carried out using the experimental set shown in FIG. 3. As is shown in FIG. 3, the experimental setup consisted of a heated bath, fiber-optic endoscope, image acquisition system, and a magnetic field source for actuating the capsules.

The experiment was carried out by immersing a porcine colon in a heated bath filled with 37° C. water. The colon, measuring approximately 4 cm in diameter, was constrained to an acrylic sheet to maintain its position and orientation underwater. This was done in order to more accurately recreate the conditions found inside a human colon with respect to temperature and pressure. A pattern of nine markers serving as fiducials, composed by three rings of three markers each, was evenly spaced and sutured throughout the lining of the colon matching the layout and placement discussed below. The device 10 was used to carry 2 mL of powdered reactants to a location approximately 4 cm past the deepest ring of markers. When the desired locus was reached, the device 10 was opened using the attractive force generated by an external magnetic field provided by a cylindrical magnet measuring 2″ in length and 2″ in diameter (K&J Magnetics, DY0Y0). Upon activation, the powdered chemicals reacted with water within the colon to produce the CO₂ responsible for insufflation. Three trials of this experiment were performed by an expert endoscopist having performed more than 2,000 procedures.

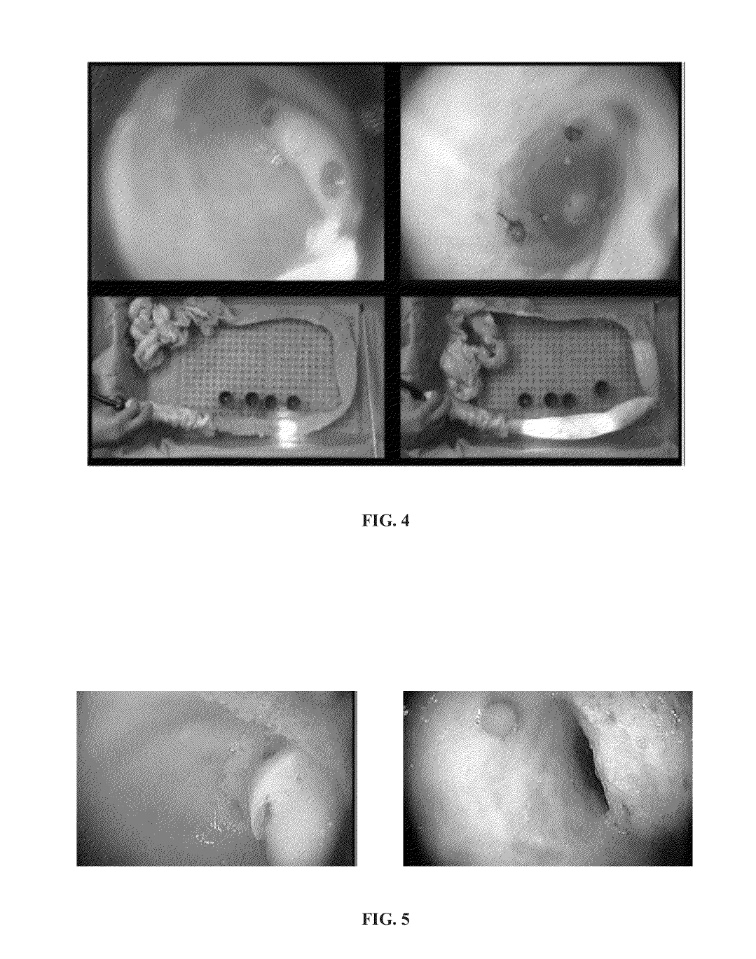

The images presented in FIG. 4 show that the device 10 was able to insufflate the section of colon to a point where most of the markers became visible. The picture presented in the upper right of FIG. 4 shows eight of the nine marks. Based on the Enhancing Visualization section below, this level of viability corresponded to roughly 350 mL of gas. While not all of the markers became visible during any one frame captured by the endoscope, based on the Enhancing Locomotion section below the level of insufflation shown in the upper right of FIG. 4 would be more than sufficient to allow a WCE to traverse the length of the inflated section of colon. Therefore, it is reasonable to expect that an actively locomoted device 10 could be used to inspect the colon and allow for visualization of all nine markers.

During the tests, a relatively large volume of foam was generated in the colon. The formation of foam is a natural byproduct of effervescent reactions however, it was interesting to note that during different runs the size of air bubbles within the foam appeared to vary, as did the time required for the bubbles to dissipate. FIG. 5 shows foam escaping from the device 10 and obscuring the view of the colon wall. While the device 10 is not visible, the image shown in the upper right of FIG. 4 also shows the formation of foam following activation of the capsule.

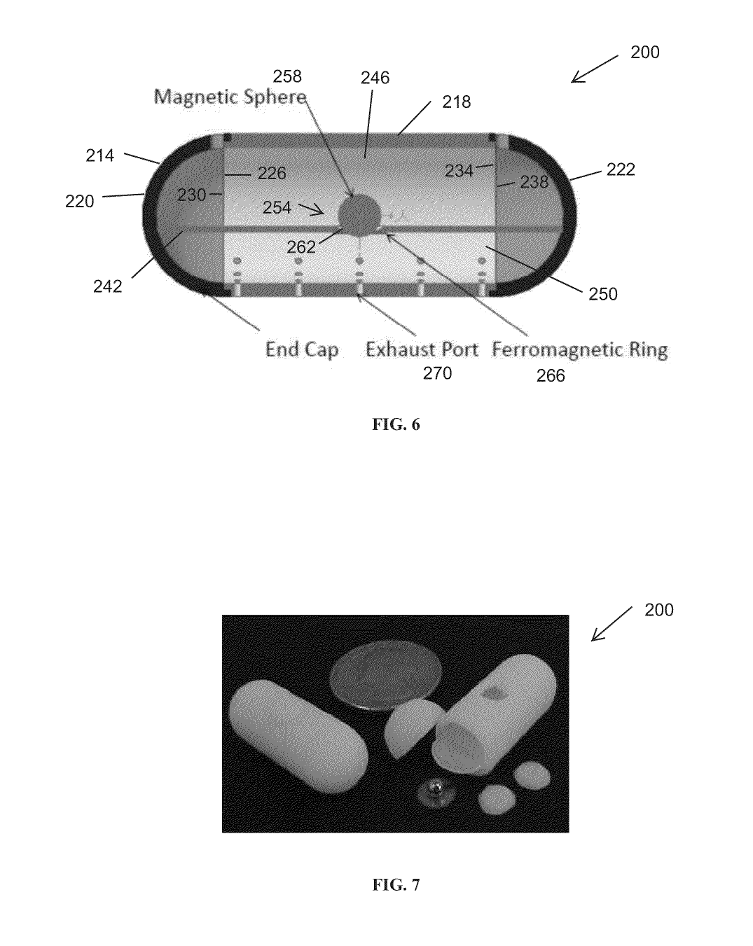

FIGS. 6-7 illustrate a wireless capsule device 200 according to another embodiment of the present invention. In this embodiment, the device 300 carries a first compound (e.g., citric acid is solid form or in solution) and a second compound (e.g., sodium bicarbonate is solid form or in solution). This design allows for the reaction to be contained within the device 200. This device 200 sacrifices some internal volume due to the transportation of H₂O, however, carrying one reactant in solution form allows metered, or throttled, control over the rate at which reactants are mixed. Hence, this device 200 sacrifices some output in order to provide control over when output can be provided.

With reference to FIGS. 6-7, the device 200 includes a housing 214 comprised of a tubular section 218, a first end cap 220, and a second end cap 222. The tubular section 218 includes a first end having a first edge 226 configured to seal with an edge 230 of the first end cap 220. The tubular section 218 also includes a second end having a second edge 234 configured to seal with an edge 238 of the second end cap 222. The housing 214 is oblong or capsular shaped as illustrated in the figures.

The device 200 also includes a divider wall 242 that extends longitudinally along a longitudinal axis thereby dividing the housing 214 into a first chamber 246 and a second chamber 250. The divider wall 242 may divide the first chamber 246 and the second chamber 250 into equal sized chambers or different sized chambers (i.e., the two chambers can comprise the same or different volumes). The divider wall 242 includes a recessed area 254 configured to receive and support a magnetic sphere 258 positioned in the first chamber 246. The recessed area 254 includes an opening 262 providing fluid communication between the first chamber 246 and the second chamber 250. The recessed area 254, the magnetic sphere 258, and the opening 262 form a ball valve.

The device 200 also includes a ferromagnetic ring 266 mounted to the divider wall 242 in the second chamber 250. The ferromagnetic ring 266 includes an opening aligned with the opening 262 in the divider wall 242. The attractive force between the ferromagnetic ring 266 and the magnetic sphere 258 keep the opening 262 closed.

The device 200 also includes a plurality of exhaust ports 270 positioned around the tubular section 218 of the housing 214. As illustrated, the exhaust ports 270 are positioned in the tubular section 218 of the second chamber 250.

The opening 262 remains closed due to the magnetic coupling generating an attractive force between the ferromagnetic ring 266 and the magnetic sphere 258. The device 200 is activated by introducing an external magnetic field strong enough to unseat the magnetic sphere 258 from the opening 262. Since the magnetic sphere 258 is free to rotate in the recessed area 254, an external magnetic field need only be a targeted distance from the device 200 as the magnetic sphere 258 will align with the orientation of the external magnetic field.

In one example, the device 200 includes dimensions of 12 mm OD, 10 mm ID, 32 mm in length with a 1 mm thick divider wall 242. Based on these dimensions, the first chamber 246 is capable of holding approximately 1 mL of citric acid solution while the second chamber 250 is capable of holding approximately 0.64 mL of base (e.g., 0.75-1.4 grams of Potassium Bicarbonate). In this example, the magnetic sphere 258 is a grade N42, ⅛″ (3.2 mm) diameter (K&J Magnetics, Inc. Model number S2) and the ferromagnetic ring 266 includes 4.173 mm OD, 1.27 mmID, and 0.1 mm thick. The external magnetic field used to actuate the magnetic sphere 258 was a 2″ diameter by 2″ thick, grade N52, axially magnetized, permanent magnet (K&J Magnetics, Inc. Model number DY0Y0-N52) applied at a distance of approximately 6 cm from the device 200.

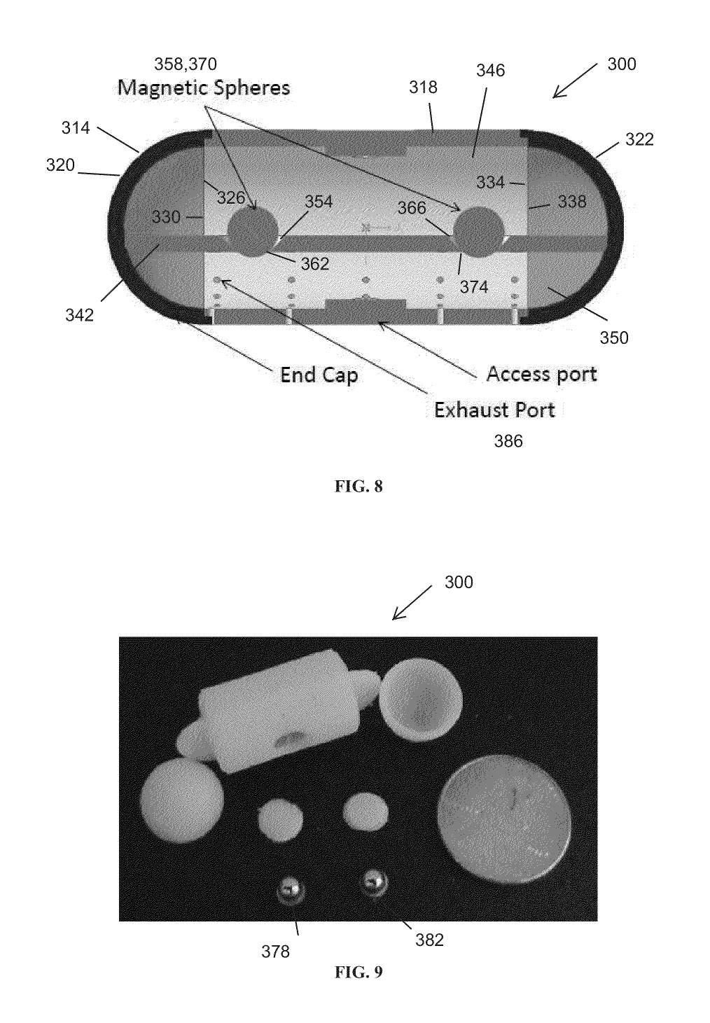

FIGS. 8-9 illustrate a wireless capsule device 300 according to another embodiment of the present invention. In this embodiment, the device 300 carries a first compound (e.g., citric acid is solid form or in solution) and a second compound (e.g., sodium bicarbonate is solid form or in solution). This design allows for the reaction to be contained within the device 300. This device 300 sacrifices some internal volume due to the transportation of H₂O, however, carrying one reactant in solution form allows metered, or throttled, control over the rate at which reactants are mixed. Hence, this device 300 sacrifices some output in order to provide control over when output can be provided.

With reference to FIGS. 8-9, the device 300 includes a housing 314 comprised of a tubular section 318, a first end cap 320, and a second end cap 322. The tubular section 318 includes a first end having a first edge 326 configured to seal with an edge 330 of the first end cap 320. The tubular section 318 also includes a second end having a second edge 334 configured to seal with an edge 338 of the second end cap 322. The housing 314 is oblong or capsular shaped as illustrated in the figures.

The device 300 also includes a divider wall 342 that extends longitudinally along a longitudinal axis thereby dividing the housing 314 into a first chamber 346 and a second chamber 350. The divider wall 342 may divide the first chamber 346 and the second chamber 350 into equal sized chambers or different sized chambers (i.e., the two chambers can comprise the same or different volumes). The divider wall 342 includes a first recessed area 354 configured to receive and support a first magnetic sphere 358 positioned in the first chamber 346. The first recessed area 354 includes a first opening 362 providing fluid communication between the first chamber 346 and the second chamber 350. The divider wall 342 also includes a second recessed area 366 configured to receive and support a second magnetic sphere 370 positioned in the first chamber 346. The second recessed area 366 includes a second opening 374 providing fluid communication between the first chamber 346 and the second chamber 350. The recessed areas 354, 366, the magnetic spheres 358, 370, and the openings 362, 374 form a first ball valve and a second ball valve.

The device 300 also includes a first ferromagnetic ring 378 mounted to the divider wall 342 in the second chamber 350. The first ferromagnetic ring 378 includes an opening aligned with the first opening 362 in the divider wall 342. The device 300 also includes a second ferromagnetic ring 382 mounted to the divider wall 342 in the second chamber 350. The second ferromagnetic ring 382 includes an opening aligned with the second opening 374 in the divider wall 342. The attractive forces between the ferromagnetic rings 378, 382 and the magnetic spheres 358, 370 keep the openings 362, 374 closed.

The device 300 also includes a plurality of exhaust ports 386 positioned around the tubular section 318 of the housing 314. As illustrated, the exhaust ports 386 are positioned in the tubular section 318 of the second chamber 350.

The openings 362, 374 remain closed due to the magnetic coupling generating an attractive force between the ferromagnetic rings 378, 382 and the respective magnetic spheres 358, 370. The device 300 is activated by introducing an external magnetic field strong enough to unseat the magnetic spheres 358, 370 from the respective openings 362, 374. Since the magnetic spheres 358, 370 are free to rotate in the respective recessed areas 354, 366, an external magnetic field need only be a targeted distance from the device 300 as the magnetic spheres 358, 370 will align with the orientation of the external magnetic field.

The device 300 can include similar dimensions to the device 200 described above.

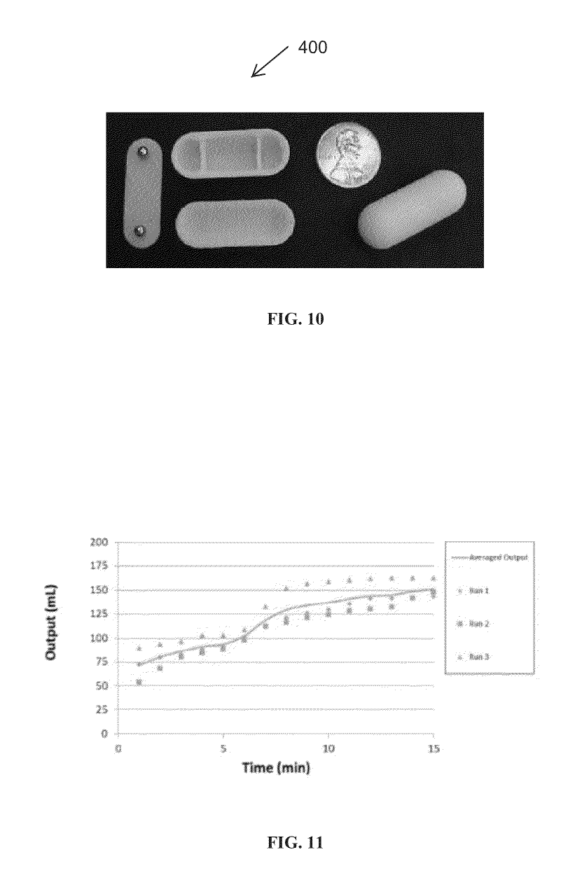

FIG. 10 illustrates a wireless capsule device 400 according to another embodiment of the present invention. Like devices 200 and 300, the device 400 carries a first compound (e.g., citric acid is solid form or in solution) and a second compound (e.g., sodium bicarbonate is solid form or in solution). This design allows for the reaction to be contained within the device 400. This device 400 sacrifices some internal volume due to the transportation of H₂O, however, carrying one reactant in solution form allows metered, or throttled, control over the rate at which reactants are mixed. Hence, this device 400 sacrifices some output in order to provide control over when output can be provided.

With reference to FIG. 10, the device 400 includes a housing 414 comprised of a first section 418 and a second section 422. The first section 418 includes a first edge 426 configured to seal with an edge 430 of the second section 422. The housing 414 is oblong or capsular shaped as illustrated in the figure.

The device 400 also includes a divider wall 442 that extends longitudinally along a longitudinal axis thereby dividing the housing 414 into a first chamber 446 and a second chamber 450. The divider wall 442 may divide the first chamber 446 and the second chamber 450 into equal sized chambers or different sized chambers (i.e., the two chambers can comprise the same or different volumes). The first section 418 includes a first partition 434 and a second partition 438 thereby separating the first chamber 446 into a first sub-chamber 444, a second sub-chamber 448, and a third sub-chamber 452.

The divider wall 442 includes a first recessed area 454 configured to receive and support a first magnetic sphere 458 positioned in the first sub-chamber 444. The first recessed area 454 includes a first opening 462 providing fluid communication between the first sub-chamber 444 and the second chamber 450. The divider wall 442 also includes a second recessed area 466 configured to receive and support a second magnetic sphere 470 positioned in the third sub-chamber 452. The second recessed area 466 includes a second opening 474 providing fluid communication between the third sub-chamber 452 and the second chamber 450. The recessed areas 454, 466, the magnetic spheres 458, 470, and the openings 462, 474 form a first ball valve and a second ball valve.

The device 400 also includes a first ferromagnetic ring 478 mounted to the divider wall 442 in the second chamber 450. The first ferromagnetic ring 478 includes an opening aligned with the first opening 462 in the divider wall 442. The device 400 also includes a second ferromagnetic ring 482 mounted to the divider wall 442 in the second chamber 450. The second ferromagnetic ring 482 includes an opening aligned with the second opening 474 in the divider wall 442. The attractive forces between the ferromagnetic rings 478, 482 and the magnetic spheres 458, 470 keep the openings 462, 474 closed.

The device 400 also includes a plurality of exhaust ports 486 positioned around the upper edge 430 of the second section 422 of the housing 314. This placement of the exhaust ports 486 allows the compound (e.g., sodium bicarbonate in solid form or in solution) in the second chamber 450 to remain therein.

The openings 462, 474 remain closed due to the magnetic coupling generating an attractive force between the ferromagnetic rings 478, 482 and the respective magnetic spheres 458, 470. The device 400 is activated by introducing an external magnetic field strong enough to unseat the magnetic spheres 458, 470 from the respective openings 462, 474. Since the magnetic spheres 458, 470 are free to rotate in the respective recessed areas 454, 466, an external magnetic field need only be a targeted distance from the device 400 as the magnetic spheres 458, 470 will align with the orientation of the external magnetic field.

The device 400 can include similar dimensions to the devices 200 and 300 described above.

Ex-Vivo Trials Using Devices 200, 300, and 400

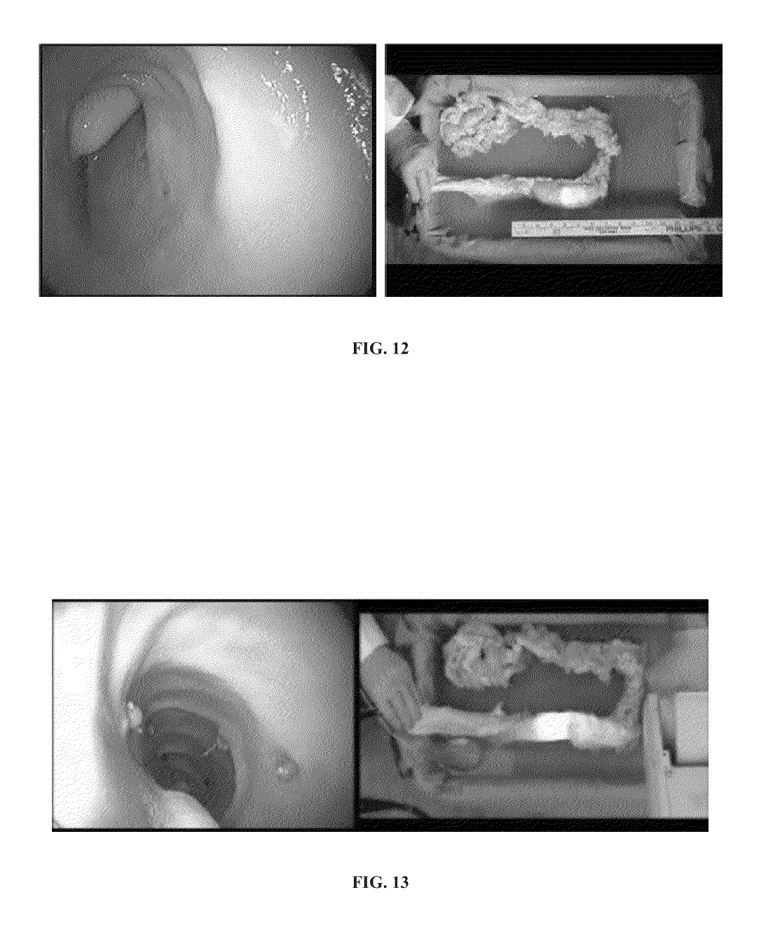

In order to assess feasibility of the devices 200, 300, 400, two ex vivo trials were undertaken. In both trials, the experimental setup shown in FIG. 3 was used. In the first trial, a single capsule was placed approximately ten centimeters past the ring of markers furthest from the rectum. A robot arm was then used to position an external permanent magnet in order to activate the capsule. The robotic arm was equipped with an ATI Nano 45 load cell to allow for force-control based manipulation of the magnet's location and orientation. In order to limit the displacement caused by the magnetic link between external permanent magnet and those used on-board the capsules, a thin piece of acrylic (approximately 3 mm in thickness) was placed on top of the heated bath. During the trials, an endoscope (Karl Storz) was used to observe the level of insufflation provided by the device and the amount of foam produced. The images presented in FIG. 12 were taken approximately two minutes after the device was activated. As can be seen in the FIG. 12, the capsule was successful in locally inflating a section of colon measuring approximately five and a half inches in length by one and a quarter inches in diameter. FIG. 12 also shows that foam generated by the capsule did not hamper the ability to view the colon lining in the area directly surrounding the capsule.

In the second trial, three capsules were placed approximately ten centimeters past the ring of markers furthest from the rectum. Once again, a robotic arm equipped with an axially-magnetized cylindrical end-effector was used to activate the capsules by simply passing over the length of the colon while remaining roughly four inches above the water level of the heated bath. FIG. 13 shows an image taken with the endoscope (left) and an exterior view of the colon (right) that were taken approximately one minute after the initial activation. As can be seen in FIG. 13, one of the capsules has been pulled away from the other two by the magnetic force developed during the initial activation. While a small amount of foam can be seen exiting the capsule, the image demonstrates that after as little as one minute the capsules were able to provide enough insufflation to allow for visualization of six markers, and two capsules, that are disbursed over a section of colon measuring approximately five inches in length.

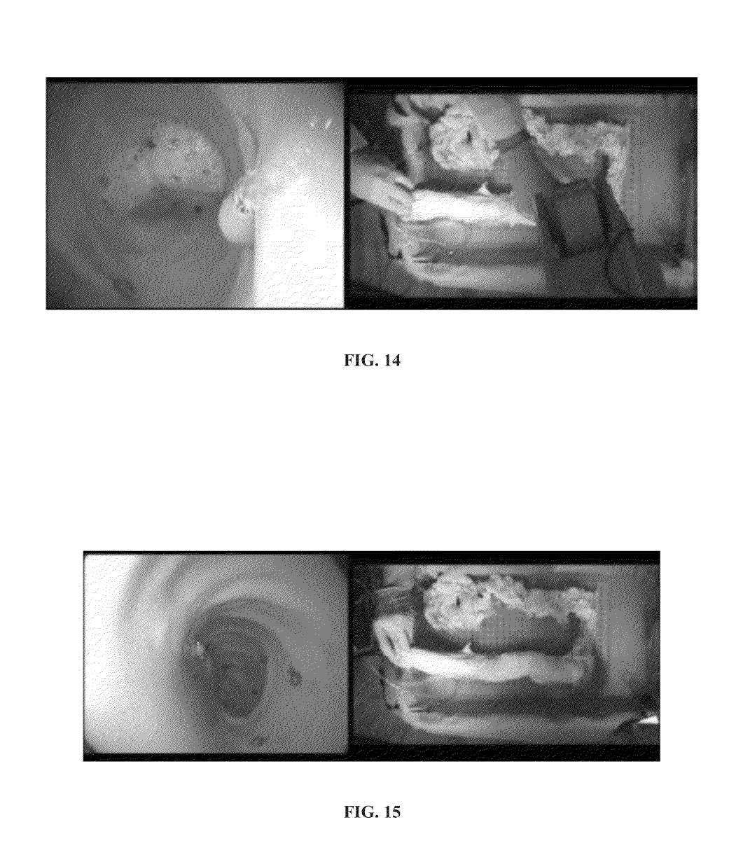

Approximately three minutes after the initial activation, the external permanent magnet was used to activate the capsules once again. During this event, the magnetic attraction developed between various components in the system caused the three capsules to come together between the second and third rings of markers. FIG. 14 shows the capsules being held against the upper side of the colon wall and the foam produced by the reaction during this dynamic event. FIG. 15 provides interior and exterior images of the colon taken approximately two minutes after the second activation of the capsules. As can be seen in FIG. 15, approximately five minutes after the initial activation, and two minutes after a subsequent activation, visualization within the colon has been greatly enhanced. This demonstrates that even after a dynamic triggering of the capsules, potassium bicarbonate and citric acid can be used to provide foam-free enhancement of visualization, and hence locomotion, within the colon.

The inventors conducted studies for establishing insufflation levels that are required for enhancing visualization of, and locomotion through, the colon during WCE for use with the devices described above. Experimental results are presented which look to quantify the amount of gas needed to enhance visualization and locomotion. This data is required to assess the feasibility of delivering a sufficient amount of gas from a given capsule with a given chemical reaction.

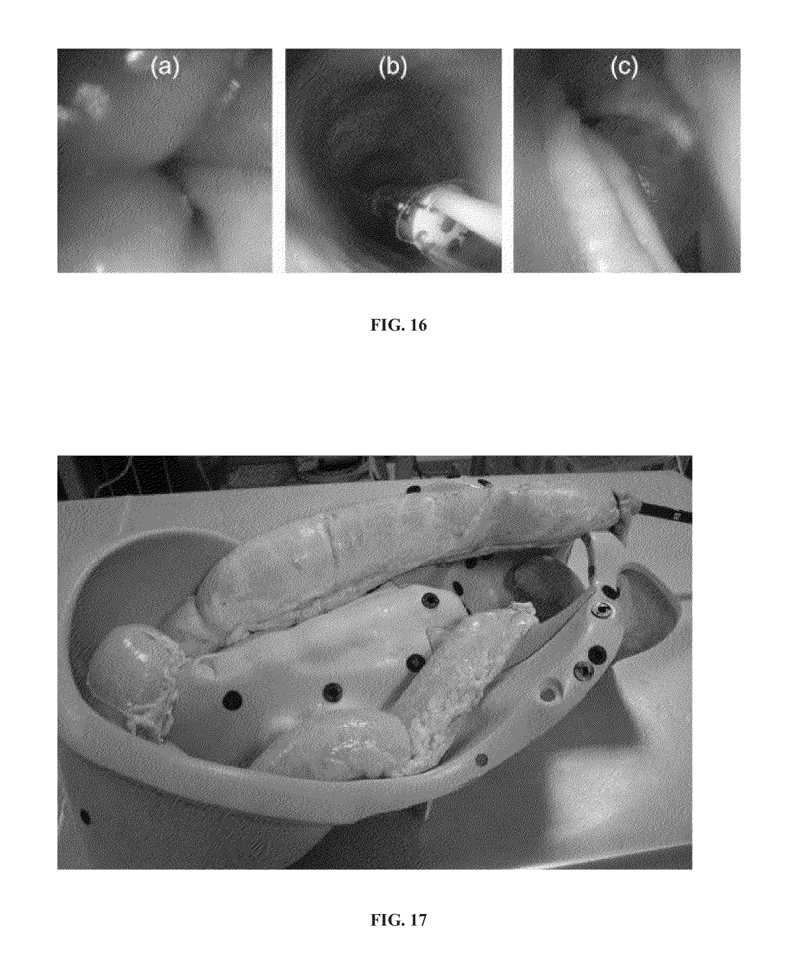

One common challenge all endoscopic capsules must contend with is the distention of tissue away from the device, and particularly away from the face of the on-board camera (see FIG. 16(a)). This is especially important in the large intestine, where the intestinal lumen is much larger than the capsule diameter. Traditional endoscopes achieve distention by inflating the intestine with air. Such insufflation provides a much clearer view of the wall of the intestine, as can be seen in FIG. 16(b). It should be noted, however, that insufflation is not without drawbacks. Inflation can cause moderate to severe pain and use of the wrong insufflating medium can result in disastrous and, at times explosive, consequences. While room air is commonly used as an insufflating medium, carbon dioxide, helium and water have also been investigated as distending mediums.

In a 2004 study presented by Burling et al., researchers found that, when using an automated CO₂ delivery system with pressure-based closed-loop control during virtual colonoscopy (rectal pressure of ≦15 mm Hg initiates insufflation while pressures ≧25 mm Hg terminate the introduction of gas), automated delivery of 1.9 L to 4.5 L (median 3.0 L) of CO₂ resulted in higher distention scores when compared to a control group which received 3.0 L of manually administered CO₂ (3.20 L (SD, 1.16 L) and 3.22 L (1.12 L) for the supine and prone scanning positions, respectively, versus 2.86 L (1.27 L) and 3.00 L (1.20 L) for the case of manual insufflation). A statistical analysis presented by Burling indicated that increased volumes of insufflation did not always result in increased distention scores, indicating that maximum distention and optimal distention are in fact not identical.

In the case of traditional colonoscopy, Bretthauer et al. found that, when instructing endoscopist to use as little insufflation as possible to achieve adequate visualization, physicians typically administered 8.3 L of CO₂ (range 1.2-19.8 L) compared to 8.2 L of air (range 1.8-18 L) with mean insufflation rates of 0.26 and 0.24 L/min, for the cases of CO₂ and room air, respectively. A similar study conducted by Leung et al. found an average 1.3 (±0.593) L of water were required to provide adequate visualization during routine colonoscopy. The difference between volumes reported by Burling, Bretthauer and Leung illustrate the vast disparity in experimental protocols and reporting conventions that currently exist in the literature.

With the average human large intestine measuring approximately 6 cm in diameter and 1.5 m in length, the total volume expected to fill a colon is on the order of 4.4 L. With Burling et al. reporting that optimal distention is slightly less than maximum distension, their numbers regarding the volume of insufflating gas used during virtual colonoscopy seem to be on par with what one might expect. Conversely, when one considers Bretthauer's et al. claim that upwards of 8 L of CO₂ or room air might be administered during a traditional colonoscopy, the reported value may seem unreasonably high. A possible explanation for this discrepancy is that Bretthauer et al. were reporting the total volumes administered, and these values do not discount volumes of gas that are withdrawn during the course of the procedure. While studies reporting the volumes of insufflation used during virtual colonoscopy may be less than half of that used during traditional colonoscopy, the use of pressure-regulating automated insufflation systems in virtual colonoscopy can result in a higher incidence of overdistention when compared to traditional colonoscopy even though the latter has been reported to use twice the volume to achieve insufflation. Regardless of the cause of the discrepancy between reported volumes of insufflating gas, the occurrence said discrepancies, and Burling's et al. observation that maximum distension is not always optimal distention, underline the fact that different CRC screening modalities require different levels of insufflation.

While a number of studies have been presented in the literature regarding the volumes of carbon dioxide or room air that are typically needed during traditional colonoscopy and virtual colonoscopy, to date, little has been published on the volumes of gas required to enhance visualization and mobility in WCE. With reports concerning the volume of gas necessary in traditional and virtual colonscopy showing dependence on the type of medium used and the manner by which insufflation is administered, the present investigation looks to experimentally evaluate the volumes of gas necessary to enhance visualization and locomotion in WCE. In the sections that follow, experimental procedures are described which look to assess the levels of insufflation necessary for enhancing visualization and mobility of wireless capsule endoscopes.

Enhancing Visualization

To determine the amount of fluid a capsule must carry in order to enhance visualization within the colon, an ex vivo experiment was performed using porcine large intestine. The experiment sought to quantify the effect insufflation has on enhancing visualization. Once relevant levels of insufflation were determined, these values can be used in conjunction with information concerning the expansion ratio produced by various chemical reactions to determine the amount of initial volume needed to produce a desired level of insufflation with a given chemical process.

In the present work, the porcine model was selected for its relative comparability to the human GI tract. The porcine model has been used to study a number of CRC screening modalities including active locomotion capsule endoscopy, virtual colonoscopy and emerging endoscope platforms.

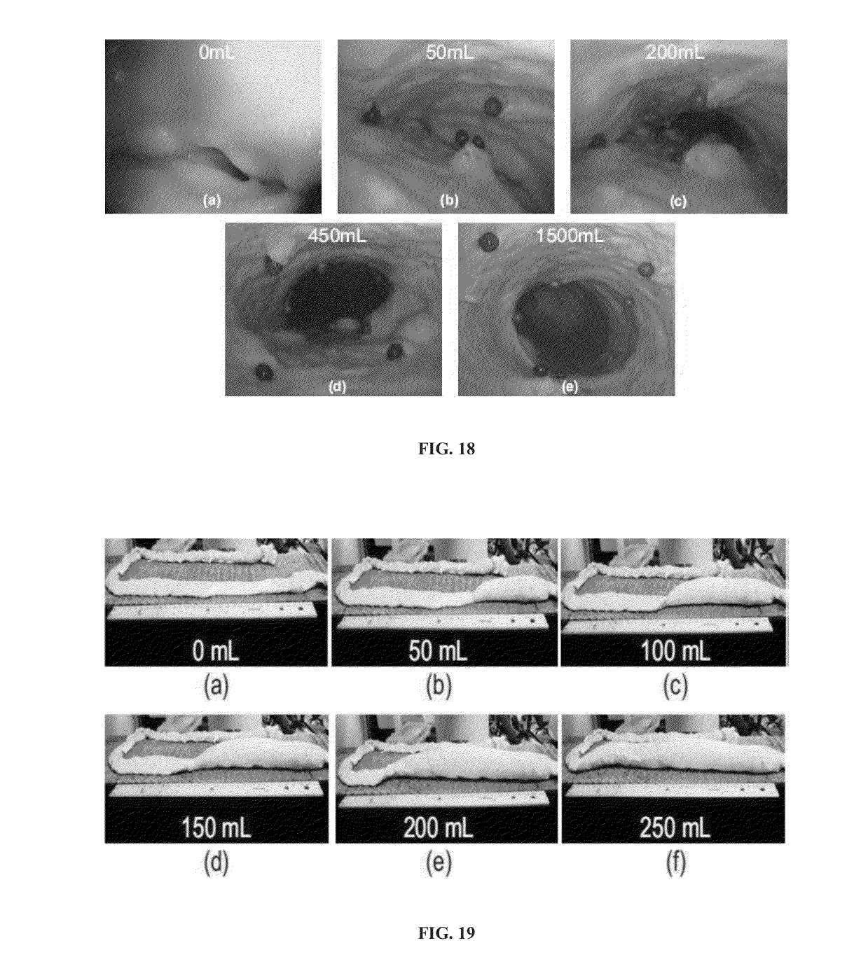

The experiment used to determine the amount of insufflation necessary to enhance visualization consisted of placing nine colored markers inside a section of intestine measuring 150 cm by 6 cm in diameter. The fiducials were evenly spaced throughout the large intestine with three markers placed around the inside diameter of the intestine and this pattern being repeated twice along the length of the intestine with approximately 3 cm between groups of markers (see FIG. 18). The deflated colon was then placed inside an anatomical model of the human torso, as shown in FIG. 17. A flexible endoscope (13803PKS endoscope, Karl Storz GmbH & Co. KG) was then placed approximately 4 cm from the first set of markers in an effort to visualize the fiducials in a manner similar to that which might be achieved using a capsule robot. A controlled air compressor was used to locally insufflate the intestine from the initially deflated state to a state where all nine markers were consistently visible by incrementing the level of insufflation 50 mL at a time. An in-line flow sensor (AWM3300V, Honeywell) was used to determine the volume of gas introduced into the intestine.

During the experiment, images were obtained at each volume increment immediately after the level of inflation was incremented and 30 seconds later in order to assess time-dependent effects. While appreciable time dependent behavior was not observed, it was interesting to note the manner by which insufflation occurred. Rather than gradually inflating the entire colon in a uniform manner, a small section surrounding the introduction site inflated first and then this inflation bubble grew along the length of the colon as additional air was introduced. Table 1 shows the number of markers that were visible at various levels of insufflation. As can be seen from the chart, all nine markers were found to be consistently visible when 450 mL of gas or more were used to insufflate the sample.

TABLE 1

| Air Volume (mL) | Markers Visible |
|---|---|
| 0 | 0 |
| 50 | 4 |
| 100 | 5 |
| 150 | 8 |
| 200 | 9 |
| 250 | 8 |
| 300 | 7 |
| 350 | 9 |
| 400 | 7 |
| 450 | 9 |
| 1500 | 9 |

Enhancing Locomotion

Wireless insufflation offers the possibility to enhance visualization for passively locomoted capsule endoscopes and actively locomoted capsules alike. However, in the latter group, wireless insufflation may actually be necessary for the platform to function at all. Due to the compliant nature of the GI lumen, active locomotion techniques like magnetic guidance often have difficulty traversing the entire length of the lumen.

In order to assess the benefit wireless insufflation might have on magnetically-actuated capsules, a second insufflation experiment was conducted using porcine large intestine, a magnetic capsule, an external magnet and robotic arm.

In this second experiment a 1.21 T NdFeB N35 permanent magnet (Sintered NdFeB magnets, B and W Technology and Trade GmbH, China) with a diameter of 60 mm, a length of 70 mm and a weight of 1.5 kg, was attached to the end effector of a 6 degree of freedom Mitsubishi RV-3S serial manipulator (Mitsubishi Electric Inc.). Three smaller internal magnets, (MTG Europe Magnet Technology Group, Germany), each having a diameter of 3 mm, a length of 10 mm, and a magnetic flux density of 1.21 T, were placed inside of a pill-sized capsule (11 mm diameter by 26 mm long). The working distance between the internal and external magnets was 150 mm. The robotic arm was preprogrammed to follow a straight path trajectory using Cosirop 2.0, a Mitsubishi Electric programming platform that allows simple functions to be written in a Basic-like language (Melfa Basic IV) and uploaded to the robotic controller by TCP/IP communication. The trajectory was 300 mm long, which approximates the length of the longest straight portion of the colon. The robot would stop its motion every 10 mm, rotate around its Z axis (roll angle) by 10 degrees, rotate around its Y axis (yaw angle) by 10 degrees, and then continue forward motion at a velocity of 5 mm/s. The rotational speed was between 5 and 10 degrees/s. This behavior was performed in order to attempt to free the capsule from the deflated lumen as a surgeon might try through teleoperation.

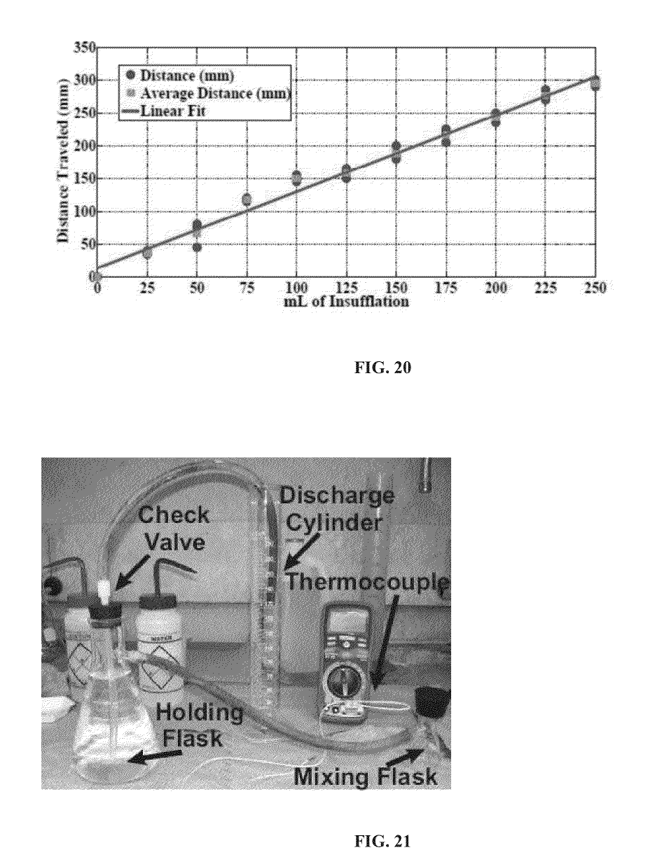

The magnetic capsule was placed inside fresh porcine large intestine (4 mm diameter), and the intestine was sealed at both ends. A 50 mL syringe was connected to a tube whose outlet was located right behind the capsule and was used to incrementally inflate the intestine in 25 mL intervals from 0 mL-250 mL. As shown in FIG. 19, this resulted in local inflation of the colon, such that the capsule could advance up until the inflation bubble ended. Three trials were performed at each insufflation interval. Results from this set of experiments are presented in FIG. 20.

Chemical Reactions for Insufflation

This section discusses various reactions that may be used for gas generation in wireless insufflation. Quantitative assessments are made to determine the relative volume each reaction might produce when initial volumes of the reactants are kept on par with the volume of commercially available capsule endoscopes. Experimental findings are used as a guide in the subsequent development of WCI devices.

Hydrogen Peroxide (H₂O₂)

Using the gas volumes reported above, we now determine the necessary fluid volume required to produce each. Hydrogen Peroxide is a promising working fluid because it can produce a large volume of gas from a small initial fluid volume. To generate gas from H₂O₂, the capsule must simply pass liquid H₂O₂ through a catalyst (e.g., a silver or platinum screen), which catalyzes the conversion to oxygen gas and water.

In order to investigate the effect H₂O₂ concentration has on the amount of gas generated by this exothermic process, known quantities of 30%, 50% and 70% solution were reacted and the amount of gas generated was recorded. The experimental setup, shown in FIG. 21, involved a mixing flask, a holding flask, and a discharge cylinder. The flasks were connected together with rubber tubing and were sealed with rubber stoppers. A thermocouple was used to measure the maximum temperature of the reaction. The catalyst used was a fine silver screen mesh, and it was cut into 11 mm circles to replicate the maximum sized screen that could fit within a swallowable capsule. An initial amount of Hydrogen Peroxide was placed in the mixing flask. The catalyst was quickly dropped into the flask, and the flask was sealed. The gas produced from this reaction was transferred to the holding flask, which held water that was displaced up a plastic tube, through a check valve, and into a graduated cylinder. The amount of water displaced corresponded with the approximate amount of gas produced from the reaction. This test was performed with initial volumes ranging from 0.5-1.25 mL (in 0.25 mL increments) for three concentrations of H₂O₂ (30%, 50%, and 70%).

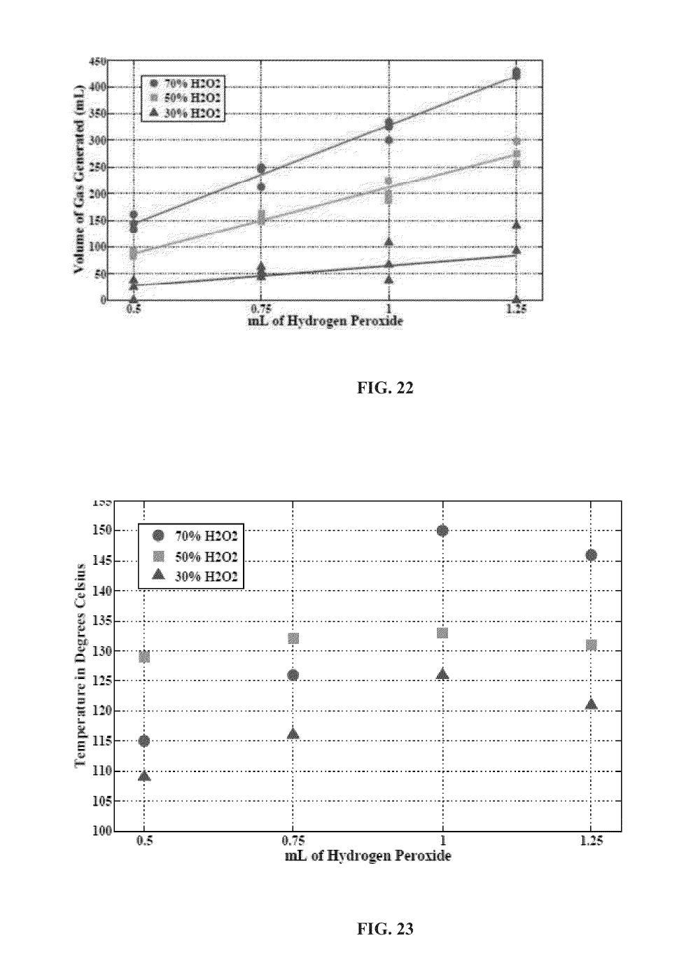

To ensure repeatability, three trials were performed for each initial volume level. One catalyst screen was used for each increment (i.e., one screen was used 3 times at 0.5 mL, and a new screen was used 3 times at 0.75 mL). The amount of water output was recorded and averaged over the three samples for each increment, at each concentration, and the results are shown in FIG. 22. The temperature on the bottom of the holding flask was measured to provide an assessment of the heat generated during decomposition and the maximum values recorded during each run are presented in FIG. 23.

Acid/Base Reactions

Acids and bases are commonly defined by the cation and anion they produce in the presence of water. When acids are added to water they produce hydrogen ions, H⁺, while bases produce hydroxide ions, OH⁻, in the presence of water. While acids reacted with some metals can be used to produce hydrogen, H_{2(g)}, they also react with compounds containing CO₃²⁻ to form water and carbon dioxide. Given the biocompatibility of this latter group of products, their use will be investigated in the present work.

In order to estimate the amount of gas a given acid/base reaction may generate we can start by determining the number of moles of each that could be delivered in a capsule of known volume.

$${V}_{tot} = {X}_{molesacid} * \frac{{MM}_{acid}}{{ρ}_{acid}} + {Y}_{molesbase} * \frac{{MM}_{base}}{{ρ}_{base}} \qquad Eq . 1$$

If the ratio of acid moles to base moles is known, equation 1 can be rewritten as

$${V}_{tot} = {X}_{molesacid} ( \frac{{MM}_{acid}}{{ρ}_{acid}} + R * \frac{{MM}_{base}}{{ρ}_{base}} ) \qquad Eq . 2$$

where R═Y_{molesbase}/moleofacid. The value of R can be determined by balancing the number of hydrogen ions, H⁺, and hydroxide ions, OH⁻, present in the initial reactants and the mass of the initial reactants can then be determined by

$$\begin{matrix}{mass}_{acid} = {MM}_{acid} \frac{{V}_{tot} {ρ}_{a} {ρ}_{b}}{{MM}_{a} {ρ}_{b} + {MM}_{b} R {ρ}_{a}} & Eq . 3 \\ {mass}_{base} = {MM}_{b} R \frac{{V}_{tot} {ρ}_{a} {ρ}_{b}}{{MM}_{a} {ρ}_{b} + {MM}_{b} R {ρ}_{a}} & Eq . 4\end{matrix}$$

If we specify a generic acid structure as HA, where A is an anion, and a generic base structure as BOH, where B⁺ is an appropriate cation, then, in generic terms, an acid/base reaction can be given as

H⁺+A+B+OH⁻→A+B+H₂O Eq. 5

where H₂O often results due to the highly favorable bonding configuration offered by H⁺+OH⁻. This pair occurs stoichiometricly when the number of H⁺ cation produced by the dissociation of HA compounds matches the number of OH⁻ anion results from the dissociation of BOH compounds. The nature of the initial HA and BOH structures will therefore have an affect on the ratio needed for stoichiometric production of H₂O, and hence of CO₂. As an example, consider the familiar vinegar and baking soda volcano. Otherwise known as an acetic acid and sodium bicarbonate volcano.

When acetic acid, CH₃CO₂H, and sodium bicarbonate, NaHCO₃, are dissolved in water they disassociate to form an acetate ion and a hydrogen ion (C₂H₃O₂⁻+H⁺) and a sodium ion, carbon dioxide and a hydroxide ion (Na⁺+CO₂+OH⁻), respectively. These reactants result in the production of sodium acetate, water and carbon dioxide. With a mole-to-mole ratio of unity, this reaction is given by

CH₃COOH+NaHCO₃→CH₃COO⁻+H⁺+Na⁺+CO₂+OH⁻ Eq. 6

→CH₃COONa+CO₂+H₂O Eq. 7

and equation 7 becomes

$${X}_{molesacid} = \frac{{V}_{tot}}{( \frac{{MM}_{acid}}{{ρ}_{acid}} + 1 * \frac{{MM}_{base}}{{ρ}_{base}} )} \qquad Eq . 8$$

Since the number of moles of CO₂ produced by this reaction is equal to the number of moles of base initially provided, the volume of the volume of CO₂ produced can be given by

$${V}_{{CO}_{2}} = \frac{{V}_{tot}}{( \frac{{MM}_{acid}}{{ρ}_{acid}} + 1 * \frac{{MM}_{base}}{{ρ}_{base}} )} R \frac{{MM}_{{CO}_{2}}}{{ρ}_{{CO}_{2}}} \qquad Eq . 9$$

If we turn our attention to the less ubiquitous citric acid and sodium bicarbonate reaction, we see that citric acid disassociates into a citric acid ion (C₆H₅O₇⁻³) and three hydrogen ions (3H⁺). When this solution is reacted with sodium bicarbonate they must be mixed in a 3-to-1 molar ratio since each sodium bicarbonate molecule will disassociate to from only one hydroxide ion. This process, which results in the production of sodium citrate (C₆H₅Na₃O₇), carbon dioxide (CO₂) and water (H₂O), is given by

C₆H₈O₇+3NaHCO₃→C₆H₅O₇⁻³+3H⁺+3Na⁺+3CO₂+3OH⁻ Eq. 10

→C₆H₅Na₃O₇+3CO₂+3H₂0 Eq. 11

While the preceding examples illustrate how the structure of the acid molecule will have a direct affect on the number of CO₂ moles produced this model does not account for the affect that properties such as density, solubility and heat of formation have on the total volume of the products. Given these unaccounted variables, experimental results are used to validate the response of the model for all possible acid/base combinations arising from the use of acetic and citric acid and sodium bicarbonate and potassium bicarbonate. Having determined the proper molar ratio for rating acetic acid with sodium bicarbonate, and citric acid with sodium bicarbonate, we now look to determine the proper ratio for stoichiometrically reacting acetic acid with potassium bicarbonate and citric acid with potassium bicarbonate.

When potassium bicarbonate is dissolved in water it disassociates to form a potassium ion (K⁺), carbon dioxide (CO₂) and a hydroxide ion (OH⁻). Due to the production of a single hydroxide ion per molecule of potassium bicarbonate, this base can be reacted in a one-to-one molar ratio with acetic acid, to give

CH₃OOH+KHCO₃→CH₃COO⁻+H⁺+K⁺+CO₂+OH⁻ Eq. 12

→CH₃COOK+CO₂+H₂O Eq. 13

or, it can be reacted in a three-to-one molar ratio with citric acid to give

C₆H₈O₇+3KHCO₃→C₆H₅O₇⁻³+3H⁺+3K⁺+3CO₂+3OH⁻ Eq. 14

→C₆H₅K₃0₇+3CO₂+3H₂O Eq. 15

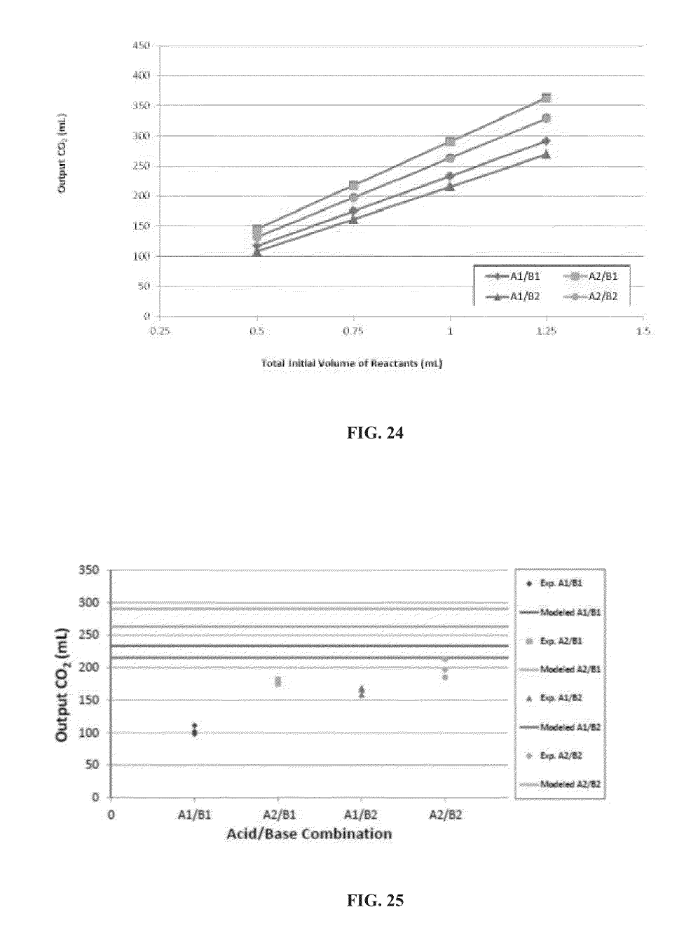

As can be seen from inspection of equations 11, 12, and 15 in the case of acetic acid being reacted with the given bases, one mole of acid results in one mole of CO₂ while in cases when citric acid is used one mole of acid results in three moles of CO₂. Given the molecular masses and densities listed in Table 2, the expected volumes produced from given initial reactant volumes can be calculated using equation 9 along with the proper molar ratio, R. Results produced by the model are shown in FIG. 24.

TABLE 2

| Chemical | Molecular Mass (g/mol) | Density (g/mL) |
|---|---|---|
| Acetic Acid | 60.05 | 1.049 |
| Citric Acid | 192.12 | 1.665 |
| Sodium Bicarbonate | 84.01 | 2.2 |
| Potassium Bicarbonate | 100.115 | 2.17 |
| Carbon Dioxide | 44.01 | 1.842 × 10⁻³ |
| dioxygen | 32 | 1.331 × 10⁻³ |

Mild acid/base reactions offer a promising method for generating relatively large volumes of gas using small initial volumes of reactants. In order to generate gas using an acid/base reaction, the reactants need only be mixed in the presence of water so as to allow their constitutive anions and cations to disassociate. While the initial volume of the reactants directly affects the total gas generated by a given acid/base reaction, the rate of reaction is restricted by the anions/cations, ability to disassociate. Hence, the rate of reaction is dependent on the volume of H₂O present when the reaction takes place.

Given the desire to generate a relatively large volume of gas in a relatively short period of time, the total volume of initial reactants must be taken into account as well as ratio of reactant to H₂O volumes. In order to investigate the use of acid/base reactions as a gas generator for wireless capsule insufflation various acid/base combinations are theoretically and experimentally evaluated. Results from these investigations are used to select a promising acid/base combination. The use of this combination is then optimized by examining the effect that the initial reactant-to-H₂O ratio has on rate of reaction and total output produced, in a given time period.

In the present investigation acetic acid, citric acid, sodium bicarbonate and potassium bicarbonate are examined as possible reactants in an acid/base gas generator. These reactants give rise to four possible acid/base combinations, as is shown in Table 3. Given a desired total initial volume, the mass of the reactants, and the resulting output, can be calculated based on the physical properties of the reactants, as is outlined in below.

TABLE 3

| | Acids | | | |
|---|---|---|---|---|
| Bases | A1 | | A2 | |
| B1 | C₂H₄O₂/NaHCO₃ | 1 | C₂H₄O₂/NaHCO₃ | 2 |
| B2 | C₂H₄O₂/KHCO₃ | 3 | C₆H₈O₇/KHCO₃ | 4 |

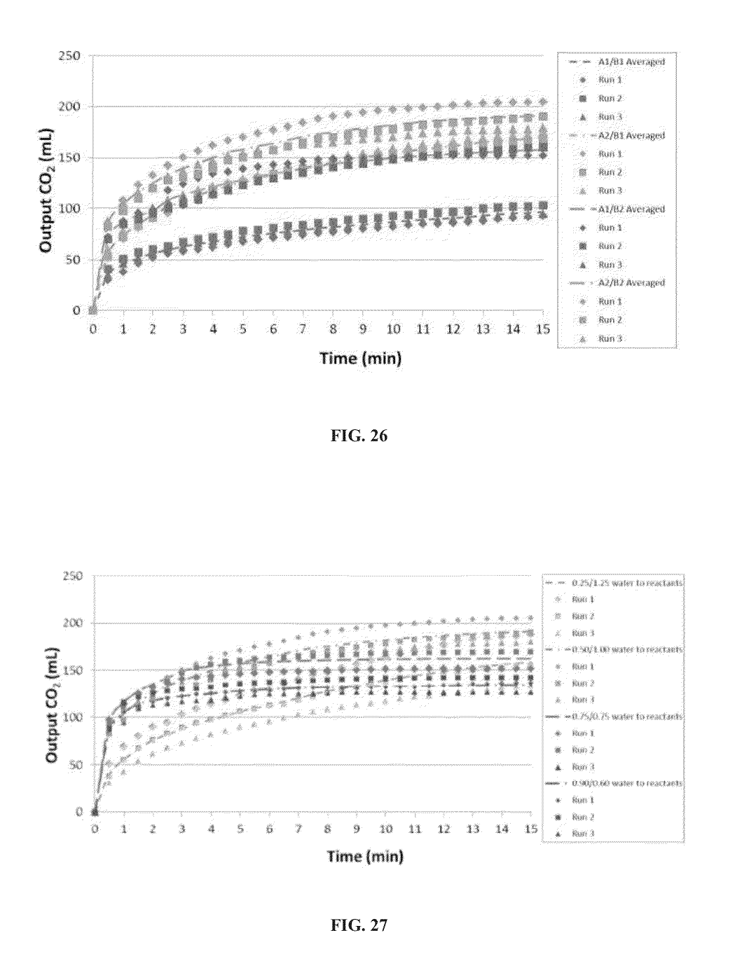

FIG. 25 shows the output produced within the first fifteen minutes when an initial volume of reactants equal to 1.0 mL is reacted in the presence of 0.5 mL of H₂O. As can be seen in FIG. 26 experimental results indicate that the combination of citric acid and potassium bicarbonate results not only in the largest average output but also the fastest rate of reaction. FIG. 26 also shows that while citric acid and sodium bicarbonate offer the second largest average output, acetic acid and potassium bicarbonate offer the second fastest rate of reaction.

Based on the results presented in FIGS. 25 and 26 the use of citric acid and potassium bicarbonate looks to offer the largest average output and fastest rate of reaction when constraints are placed on total initial volume of the reactants. In order to determine the effect that initial water volume has on rate of reaction and total output generated by this acid/base combination, additional experiments were performed with initial water to reactant volumetric ratios of 0.25/1.25 and 0.75/0.75 mL per mL. Results from these tests are presented, along with the case corresponding to an initial water to reactant volumetric ratio of 0.5/1, in FIG. 27.

The results presented in FIG. 27 indicate that while the relative volume of water present at the start of the reaction has an effect on the total output produced from an initial quantity of reactants (i.e., more water in a given initial volume means less reactants and hence less output) they also illustrate the pronounced affect this variable has on the initial rate of reaction (i.e., the amount of output produced during the first moments of the reaction). With an average colonoscopy taking between 30 minutes to an hour to complete, a wireless insufflation capsule should be capable of generating the necessary volume of gas within some fraction of this time in order to keep WCE-based colonoscopy times on par with those conducted using traditional endoscopy. Based on the results presented in FIG. 27 it appears as though, for a given total initial volume, a tradeoff exists between the amount of output that can be generated in a given time and total output that might be expected as t→∞. As can be seen from the data presented in FIG. 27, a water-to-reactant volume ratio of approximately one-half-to-one seems to offer the best compromise between fast rate of reaction and total volume produced. If we redefine the examined water-to-reactant volume ratios in terms of initial grams of citric acid per initial volume of H₂O, the data presented in FIG. 27 is classified by dilution as 3.78, 1.51, 0.75 and 0.50 grams of citric acid per milliliter of H₂O. It is interesting to note, the case corresponding to an initial water-to-reactant volume of 1-to-1 provides a level of dilution that approximately matches reported values for the solubility of citric acid.

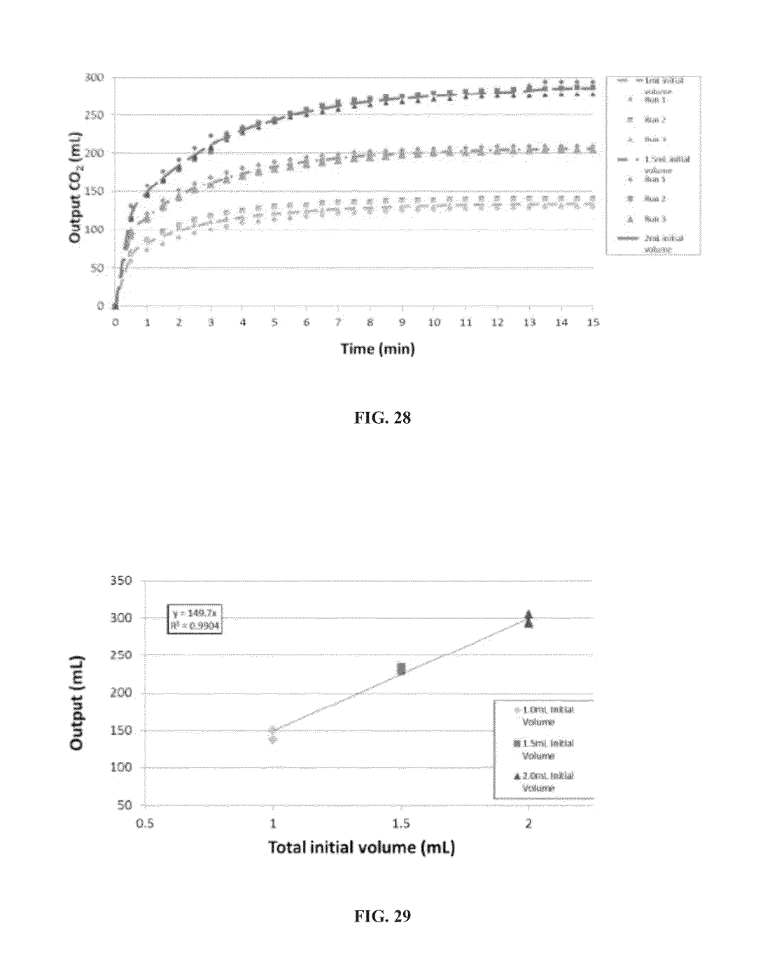

Using the information provided in FIG. 27, a final set of experiments was conducted to determine the output generated when total initial volume is set equal to 1.0, 1.5 and 2.0 mL and the water-to-reactant ratio is selected based on the results presented in FIG. 27. These initial volume levels were selected based on the typical size of commercially available capsule endoscopes (2.47 mL) and the likely usable volume within a capsule of said dimension. The transient output produced from said initial volumes are presented in FIG. 28. FIG. 29 shows the average output produced within the first ten minutes of reacting said initial volumes of acid, base and H₂O. The linear trend presented in FIG. 29 was extrapolated to determine the initial volume of reactants needed to produce desired volumes of CO₂. Results of the extrapolation are presented in Table 4.

TABLE 4

| CO₂ (mL) | C₆H₈O₇ + KHCO₃ (mL) | C₆H₈O₇ + KHCO₃ + H₂O) (mL) |
|---|---|---|
| 50 | 0.22 | 0.33 |
| 100 | 0.45 | 0.67 |
| 150 | 0.67 | 1.00 |
| 200 | 0.89 | 1.34 |
| 250 | 1.11 | 1.67 |
| 300 | 1.33 | 2.00 |
| 350 | 1.56 | 2.34 |
| 400 | 1.78 | 2.67 |
| 450 | 2.00 | 3.00 |
| 1500 | 6.68 | 10.02 |

Based on the trend depicted in FIG. 29, one might reasonably expect to obtain 450 mL from a capsule containing powdered acid and base reactants or 300 mL from a capsule containing reactants plus H₂O such that the initial water-to-reactant volume ratio is approximately one-half-to-one.

Various features and advantages of the invention are set forth in the following claims.

What is claimed is:

1. A device for insufflating a body cavity, the device comprising:
a first chamber, a second chamber, and a port;
a wall between the first chamber and the second chamber, the wall including a recessed portion and an opening in the recessed portion for fluid communication between the first chamber and the second chamber;
a magnetic sphere positioned in the first chamber and configured to be received in the recessed portion and to close the opening; and
a ferromagnetic ring positioned in the second chamber, the ferromagnetic ring including an opening aligned with the opening in the recessed portion, the ferromagnetic ring magnetically coupled to the magnetic sphere.

2. The device of claim 1 further comprising a first compound contained within the first chamber.

3. The device of claim 2 further comprising a second compound contained within the second chamber.

4. The device of claim 3 wherein the first compound is citric acid and the second compound is sodium bicarbonate and wherein a molar ratio of the first compound to the second compound is between about 4:1 to about 2:1.

5. The device of claim 3 wherein the first compound is citric acid and the second compound is sodium bicarbonate and wherein a molar ratio of the first compound to the second compound is about 3:1.

6. The device of claim 2 wherein the first compound is citric acid.

7. The device of claim 6 wherein the citric acid is in solid form.

8. The device of claim 3 wherein the second compound is sodium bicarbonate.

9. The device of claim 8 wherein sodium bicarbonate is in solution.

10. The device of claim 1 wherein a first compound is mixed with a second compound upon activation of the device to thereby produce a sufficient amount of carbon dioxide (CO₂) that is expelled through the port to insufflate the body cavity.

11. The device of claim 3 wherein the first compound is citric acid and the second compound is sodium bicarbonate.

12. The device of claim 11 wherein the citric acid is in solid form and the sodium bicarbonate is in solution.

13. The device of claim 1 further comprising a plurality of ports arranged semi-circumferentially on the second chamber.

14. The device of claim 1 further comprising a plurality of ports arranged in a mid-section on the second chamber.

15. The device of claim 1 wherein the wall includes a second recessed portion and a second opening in the second recessed portion for fluid communication between the first chamber and the second chamber, and further comprising a second magnetic sphere positioned in the first chamber and configured to be received in the second recessed portion and to close the second opening, and further comprising a second ferromagnetic ring positioned in the second chamber, the ferromagnetic ring including an opening aligned with the second opening in the second recessed portion, the second ferromagnetic ring magnetically coupled to the second magnetic sphere.

16. The device of claim 1 wherein the device is activated by a magnet positioned external to the body cavity to overcome the magnetic coupling between the magnetic sphere and the ferromagnetic ring.

17. The device of claim 3 wherein a majority of the chemical reaction between the first compound and the second compound to generate CO₂ occurs in the first and second chambers.

18. A device for insufflating a body cavity, the device comprising:
a first chamber including citric acid;
a second chamber including sodium bicarbonate;
a port in one of the first chamber and the second chamber; and
a mixing chamber that combines the sodium bicarbonate and the citric acid to produce a sufficient amount of carbon dioxide (CO₂) through the port to insufflate the body cavity upon activation of the device.

19. The device of claim 18 further comprising a wall between the first chamber and the second chamber, the wall including a first recess configured to support a first magnet and a first opening formed through the first recess, wherein the wall includes a second recess configured to support a second magnet and a second opening formed through the second recess, and wherein a magnetic force maintains the first magnet and the second magnet in their respective recesses such that fluid communication is prohibited between the first chamber and the second chamber.

20. The device of claim 19 wherein the device is activated by a third magnet positioned external to the body cavity to overcome the magnetic force and to displace the first magnet and the second magnet and allow fluid communication between the first chamber and the second chamber.

21. The device of claim 18 wherein a molar ratio of the citric acid to the sodium bicarbonate is between about 4:1 to about 2:1.

22. The device of claim 18 wherein a molar ratio of the citric acid to the sodium bicarbonate is about 3:1.

23. The device of claim 18 wherein the citric acid is in solid form.

24. The device of claim 18 wherein the sodium bicarbonate is in solution.

25. A method of insufflating a body cavity, the method comprising:
positioning a swallowable device in a body cavity;
activating the device within the body cavity by applying a magnetic field near the device;
admixing sodium bicarbonate and citric acid in the capsule; and
producing a sufficient amount of carbon dioxide (CO₂) due to the chemical reaction between the sodium bicarbonate and citric acid to insufflate the body cavity.

26. The method of claim 25 further comprising moving a magnet from a first position to a second position with the magnetic field to provide fluid communication between a first chamber and a second chamber upon activation of the device.

27. The method of claim 25 further comprising expelling the carbon dioxide through a port in the device and into the body cavity.

28. The method of claim 25 wherein a molar ratio of the citric acid to the sodium bicarbonate is between about 4:1 to about 2:1.

29. The device of claim 25 wherein a molar ratio of the citric acid to the sodium bicarbonate is about 3:1.
